(19) United States
(12) Patent Application Publication
(10) Pub. No.: US 2014/0277019 A1
(43) Pub. Date: Sep. 18, 2014

(54) SURGICAL ACCESSORY FOR USE IN IMPLANTING MEDICAL DEVICE

(71) Applicant: NeuroPace, Inc., Mountain View, CA (US)

(72) Inventor: John Dunagan PEARSON, Mountain View, CA (US)

(73) Assignee: NeuroPace, Inc., Mountain View, CA (US)

(21) Appl. No.: 14/197,449

(22) Filed: Mar. 5, 2014

Related U.S. Application Data
(60) Provisional application No. 61/789,952, filed on Mar. 15, 2013.

(51) Int. Cl. A61B 17/17

(52) U.S. Cl. CPC A61B 17/1739
USPC 606/167

(57) ABSTRACT

A surgical accessory for use in implanting a medical device in a cranial bone of a human patient may serve as a template during a craniectomy for cutting a hole in which a ferrule for a medical device may be situated and then secured. The surgical accessory is dimensioned to allow a surgeon to gauge how far above the outer surface of the cranium the medical device will extend once the ferrule has been secured to the outer bone table and the medical device has been cradled in the ferrule.

### CROSS-REFERENCE TO RELATED APPLICATION(S)

This application claims the benefit of U.S. Provisional Application Ser. No. 61/789,952 entitled “Surgical Accessory for Use in Implanting Medical Device,” filed Mar. 15, 2013, which is expressly incorporated by reference herein in its entirety.

### BACKGROUND

1. Field

The present technology relates generally to surgical accessories that are useful during the implantation of medical devices, especially for the implantation of medical devices in a patient's cranial bone.

2. Background

Implantable medical device systems are known that include implantable components such as one or more sensing and/or stimulation leads and an active medical device, such as neurostimulator, to which the leads may be connected. In some cases, the cranially-implanted component is situated in a hole formed in the patient's skull by a craniectomy. A ferrule or tray may be secured in the hole in which the neurostimulator can then be placed.

### SUMMARY

A surgical accessory for use in implanting a medical device in a cranial bone of a human patient includes a body dimensioned to approximate the dimensions of an implantable medical device. The dimensions may include at least a depth, an outer perimeter, and a top surface of the implantable medical device. The accessory also includes a pair of recessions provided in the top surface of the body. The recessions are configured to receive a surgeon's fingers or a surgical tool for grasping the body of the surgical accessory. The surgical accessory may be configured to fit within a ferrule adapted to receive the implantable medical device and to be situated in a hole formed in the cranial bone of the human patient.

A method for using a surgical accessory in a procedure involving a cranially-implantable medical device includes tracing an outline of the surgical accessory on a top surface of the patient's cranium, forming a hole in the cranium extending from an outer bone table through to an inner bone table of the cranium based on the outline, and situating a ferrule in the hole. The ferrule may be provided with at least one bendable tab. In this case, situating the ferrule may include bending the at least one tab to adjust the depth to which the ferrule extends into the hole. The method also includes placing the surgical accessory within the ferrule and assessing the extent to which a top surface of the surgical accessory is or is not approximately flush with the outer bone table. The method further includes adjusting, if necessary, the depth of the ferrule until the top surface of the surgical accessory when placed in the ferrule is approximately flush with the outer bone table. The surgical accessory is provided with a body dimensioned to approximate the dimensions of an implantable medical device including at least a depth, an outer perimeter and a top surface of the implantable medical device. The surgical accessory may include a pair of recessions in the top surface of the body that are configured to receive a surgeon's fingers or a surgical tool for grasping the body of the surgical accessory as necessary while it is being manipulated relative the patient. The pair of recessions may be used to manipulate the surgical accessory relative to the cranium and the ferrule.

A surgical accessory device for use in implanting a medical device in a cranial bone of a human patient includes a body having a top surface. The body is dimensioned to approximate the form factor of an implantable medical device. The accessory device includes at least one recession provided in the top surface that is configured to receive a surgeon's fingers or a surgical tool. The form factor is characterized by one or more of a depth, an outer perimeter and curvature. The surgical accessory is further configured to fit within a ferrule adapted to receive the implantable medical device.

### BRIEF DESCRIPTION OF THE DRAWINGS

The accompanying drawings, which are incorporated in and form a part of this specification, illustrate and serve to explain the principles of embodiments in conjunction with the description. Unless specifically noted, the drawings referred to in this description should be understood as not being drawn to scale.

FIG. 1 is a schematic view of a patient's skull in which a craniectomy has been performed to create a fenestration or hole in which to implant a medical device.

FIG. 2 is a schematic view of a user/surgeon securing a ferrule or cradle or tray in the fenestration or hole of FIG. 1 to receive a medical device.

FIG. 3 is a side elevational view of a surgical accessory for use in implanting a medical device in a patient's cranium.

FIG. 4 is a cross-sectional view of the surgical accessory of FIG. 5, taken along the line A-A of FIG. 5.

FIG. 5 is a top plan view of the surgical accessory of FIG. 3.

FIG. 6 is a perspective view of the surgical accessory of FIG. 3.

FIG. 7 is a perspective view of a medical device installed in a ferrule following a procedure to implant the medical device in a patient's cranium.

### DETAILED DESCRIPTION

Various aspects of the disclosure will be described more fully hereinafter with reference to the accompanying drawings. This disclosure may, however, be embodied in many different forms by those skilled in the art and should not be construed as limited to any specific structure or function presented herein. Rather, these aspects are provided so that this disclosure will be thorough and complete, and will fully convey the scope of the disclosure to those skilled in the art. Based on the teachings herein, one skilled in the art should appreciate that the scope of the disclosure is intended to cover any aspect of this disclosure, whether implemented independently of or combined with any other aspect of the disclosure. For example, an apparatus may be implemented or a method may be practiced using any number of the aspects set forth herein. In addition, the scope of the disclosure is intended to cover such an apparatus or method which is practiced using other structure and/or functionality in addition to or instead of other aspects of this disclosure. It should be understood that any aspect of the disclosure disclosed herein may be embodied by one or more elements of a claim.

An implantable medical device system may include implantable components including one or more electrode-bearing brain leads for delivering stimulation to (or for sensing field potential measurements from) neural tissue, and an active implantable medical device configured to deliver stimulation signals through the electrodes and leads and/or to receive and process physiological signals sensed by the electrodes from the patient (e.g., EEG signals). The lead(s) may be connected to the neurostimulator at a lead connector associated with a housing of the neurostimulator.

External components of an implantable neurostimulation system may include a “programmer” that a user/physician can use to communicate bidirectionally with the implantable medical device (e.g., to program the implant with parameters that govern what stimulation waveforms the implant will generate and when it will generate them and through which electrodes the stimulation waveforms will be delivered). The programmer and implant also may be configured so that information acquired by and/or stored in a memory of the implant can be downloaded to the programmer for analysis by a physician. An additional external component may include a patient remote monitor, which may be configured for one-way communication with the implant (e.g., to download data from the implant onto the patient remote monitor). Another external component may include a central database with which the programmer and any patient remote monitor can be networked. A responsive neurostimulation system manufactured under the tradename “RNS SYSTEM” by NeuroPace, Inc. is an example of such an implantable medical device system.

Implantable medical device systems including a cranially-implanted component also are described in, for example, U.S. Pat. No. 6,016,449 to Fischell, et al. for “System for Treatment of Neurological Disorders”, issued Jan. 18, 2000, U.S. Pat. No. 6,810,285 to Pless et al. for “Seizure Sensing and Detection Using An Implantable Device,” issued Oct. 24, 2004, and U.S. Pat. No. 6,690,974 to Archer et al. for “Stimulation Signal Generator for an Implantable Device” issued Feb. 10, 2004. Each of the '449, '285 and '974 patents is hereby incorporated by reference in the entirety.

To ready a patient for the medical device to be implanted in the cranium, and referring now to FIG. 1, a craniectomy may be performed to remove a portion of the bone in the patient's cranium 102. The cranial bone surrounding the portion removed by the craniectomy may be characterized as having a depth D_{c}, measured between a top surface of the patient cranium (outer bone table) and an inner surface of the patient's cranium (inner bone table). It will be appreciated that the depth D_{c} may vary from patient to patient, but an average depth D_{c} for a class of patients (e.g., adult males, children, etc.) may be available. The medical device may be intended to be implanted in the hole or fenestration 110 left behind after the removal of the bone. FIG. 7 is an illustration of a medical device 710 implanted in a hole 110 formed in a cranium 202.

Once the hole 110 has been formed, and referring now to FIG. 2, a ferrule or cradle or tray 210 is situated in the hole 110. Desirably, the ferrule 210 is made of biocompatible metal such as titanium. The ferrule 210 has a body 220 and may be characterized by a ferrule a ferrule depth D_{F}. The ferrule 210 may be provided with one or more tabs 215 which are flexible or bendable so that once the ferrule 210 is placed in the hole 110, each tab 215 can be manipulated towards the top surface of the cranium and used to secure the ferrule 210 to the skull. Each tab 215 may be provided with an aperture 217 for receiving a bone-attaching element such as a bone screw (not shown). In FIG. 2, the ferrule 210 is provided with four tabs 215 each having an aperture 217 for receiving a bone-attaching element. The ferrule 210 extends from a top surface of the cranial bone in towards the brain by a ferrule depth D_{F}.

The ferrule 210 is configured to receive the housing of the medical device that is to be implanted in the cranium 102, e.g., a housing of a neurostimulator. The neurostimulator may be characterized by an average depth D_{N}. The positioning of the ferrule 210 in the hole 110 will determine how much of the device depth D_{N} will extend above the bone table when the ferrule has been secured to the cranium 102 and the neurostimulator has been situated in the ferrule.

The tabs 215 of the ferrule 210 may be intended to be bent by the neurosurgeon at the time the ferrule is situated in the cranial hole 110 in a manner customized for the anatomy of the particular patient. The location of the bend in each ferrule tab 215 is directly responsible for the extent to which the device depth, D_{N} will extend about the top surface of the cranium 102. Put another way, the height of the device that extends above the outer bone table can be controlled to some degree by the surgeon's manipulation and placement of the ferrule tabs 215.

If a ferrule tab 215 is bent close to the ferrule body 220, the ferrule 210 will hold the implantable medical device higher relative to the outer bone table than if a ferrule tab 215 is bent further from the ferrule body 220. Each of the four ferrule tabs 215 is bent independently and the bend location can be the same or different as any other ferrule tab. Since the medical device (e.g., the neurostimulator) nests within the ferrule (like a cradle), and the location (height) of medical device-contacting surfaces of the ferrule 210 relative to the outer bone table (top surface of the cranium 102) can be controlled using the ferrule tab 215 bend locations, the ferrule 210 determines the implanted height of outward- (scalp-) facing surfaces of the implantable medical device relative to the bone table (surface of the cranium).

The implantable medical device may have flexible or rigid components (such as leads, strain reliefs, antennae, re-charging coils, etc.) which are intended to rest flush on the bone table (surface of the cranium, as defined previously). There may be an ideal “device installation height” where any flexible or rigid component designed to rest on the bone table has a smooth, continuous transition onto the bone table from the device within the ferrule 210.

If the “device installation height” is higher than the ideal height, there may be gaps under the flexible or rigid components designed to rest on the outer bone table. This can cause irritation or erosion of the scalp which can lead to infection. Further, gaps under components can increase the risk of movement and fatigue of the flexible or rigid components designed to rest on the bone table.

If the “device installation height” is lower than the ideal height, the flexible or rigid components designed to rest on the bone table may interfere with the bone table, preventing the implantable medical device from nesting as designed into the ferrule 210. The transition of flexible components designed to rest on the outer bone table may be too abrupt, increasing the risk of kinking, bending, or otherwise stressing the components between the device in the ferrule 210 and the outer bone table. Increased pressure may be exerted on the scalp by flexible or rigid components intended to rest on the outer bone table, which can cause irritation or erosion of the scalp which can lead to infection.

Referring now to FIGS. 3-6, a surgical accessory according to embodiments comprises an implant guide tool 300 that serves multiple purposes during the process of implanting the cranially-mounted medical device. In one configuration, the implant guide tool approximates the form factor of the implantable device that is to be implanted. “Form factor” as used herein refers to the physical size and shape of the implantable device. The form factor may be characterized by the dimensions of the device, including for example, at least the depth or thickness of the device, and the outer perimeter. The form factor may also be characterized by a contour or curvature.

After determining the area on the cranium where the ferrule and implantable neurostimulator are to be implanted, the implant guide tool 300 is used as a stencil or template where a line is marked or scribed around the perimeter of the implant guide tool 300 to indicate the area inside which bone is to be removed in the shape of the ferrule 210 which is to be installed there. The surgeon can use the craniectomy template as a guide to determine when sufficient bone has been removed such that the ferrule can fit in the hole formed by the craniectomy.

Once the craniectomy is cut in the cranium, the implant guide tool 300 can be used in conjunction with the ferrule 210 (prior to, during, and after the tabs 215 are bent) to indicate the “device installation height” of the device relative to the ferrule 210. An outward- (scalp-) facing surface 320 of the implant guide tool 300 may be designed to match exactly the device curvature. Further, with the implant guide tool 300 in the ferrule 210 and the tabs 215 bent in any proposed configuration by the surgeon, a smooth transition from the implant guide tool 300 to the outer bone table (no step up or step down) will indicate to the surgeon that the “device installation height” will be close to the ideal (not higher or lower). By examining the height continuity of the transition of the implant guide tool 300 to the outer bone table (lack of step up or step down), the ferrule tabs 215 can be re-bent as necessary until the desired device installation height is achieved. This may help to ensure a correct positioning of the flexible or rigid device components intended to rest on the outer bone table.

The implant guide tool 300 also may be installed in a ferrule 210 and used as a cranial prosthesis in the event a medical device installed in the cranial hole 110 either has to be removed (e.g., so that it can be replaced with another device in a subsequent procedure) or in the event the medical device is never installed for some reason (medical device becomes contraindicated for the patient for some reason).

In sum, the implant guide tool 300 allows the surgeon to determine the dimensions of the hole 110 to be formed during the craniectomy to install the medical device. The implant guide tool 300 allows the surgeon to check the extent to which the medical device will extend about the outer bone table using a combination of the implant guide tool 300 and the ferrule 210 before taking the medical device out of its sterile packaging, which help to minimize the extent to which the surgeon must handle the medical device prior to installing it in the ferrule 210 and, for example, connecting it to any lead or leads. Put another way, the surgeon may use the implant guide tool 300 as a substitute for the actual medical device while deciding whether he or she has achieved the right fit for the ferrule 210 and the device in the hole 110.

The implant guide tool 300 according to embodiments may be formed of a suitable biocompatible material such as poly-ether-etherketone (PEEK), the material manufactured under the tradename “ULTEM” or other suitable medical grade plastic and can be rendered convenient for the surgeon to handle. The implant guide tool 300 is designed to be have features such as handles with one or more relatively large openings or recession 330 (e.g., two openings or recessions dimensioned so as to each be large enough to accept a surgeon's gloved finger are shown in FIGS. 5 and 6). The recessions 310 makes it easier for a surgeon to manipulate the implant guide tool 300 in a typical surgical environment in which the tool is intended to be used.

The process for making the implant guide tool 300 may be uncomplicated and straightforward, such as injection molding. The implant guide tool 300 has the added advantage of being usable as a suitable prosthesis for the cranial bone excised by the craniectomy, in the event the medical device is not, in fact, implanted in the patient or in the event a previously-installed medical device is explanted from the patient.

As disclosed herein, a surgical accessory for use in implanting a medical device in a cranial bone of a human patient includes a body dimensioned to approximate the dimensions of an implantable medical device including at least a depth, an outer perimeter and a top surface of the implantable medical device. The accessory also includes a pair of recessions provided in a top surface of the body configured to receive a surgeon's fingers or a surgical tool for grasping the body of the surgical accessory as necessary while it is being manipulated relative the patient. The surgical accessory may further configured to fit within a ferrule that is adapted to receive the implantable medical device and to be situated in a hole formed in the cranial bone of the human patient. The surgical accessory may be formed from a medical grade plastic. The surgical accessory may be manufactured with an injection molding process.

As disclosed herein, a method related to placement of a cranially-implantable medical device uses a surgical accessory provided with a body dimensioned to approximate the dimensions of an implantable medical device including at least a depth, an outer perimeter and a top surface of the implantable medical device. The method includes tracing an outline of the surgical accessory on a top surface of the patient's cranium, forming a hole in the patient's cranium extending from the outer bone table through to the inner bone table of the cranium based on the outline, situating a ferrule in the hole. The ferrule may be provided with at least one bendable tab. As such, situating the ferrule may further include bending the at least one tab to adjust the depth to which the ferrule extends into the hole, placing the surgical accessory within the ferrule and assessing the extent to which a top surface of the surgical accessory is or is not approximately flush with the outer bone table, and adjusting, if necessary, the depth of the ferrule until the top surface of the surgical accessory when placed in the ferrule is approximately flush with the outer bone table. The surgical accessory may include a pair of recessions provided in a top surface of the body configured to receive a surgeon's fingers or a surgical tool for grasping the body of the surgical accessory as necessary while it is being manipulated relative the patient. In this case, the method may include using the pair of recessions to manipulate the surgical accessory relative to the cranium and the ferrule.

The various aspects of this disclosure are provided to enable one of ordinary skill in the art to practice the present invention. Various modifications to exemplary embodiments presented throughout this disclosure will be readily apparent to those skilled in the art, and the concepts disclosed herein may be extended to other magnetic storage devices. Thus, the claims are not intended to be limited to the various aspects of this disclosure, but are to be accorded the full scope consistent with the language of the claims. All structural and functional equivalents to the various components of the exemplary embodiments described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112, sixth paragraph, unless the element is expressly recited using the phrase “means for” or, in the case of a method claim, the element is recited using the phrase “step for.”

1. A surgical accessory for use in implanting a medical device in a cranial bone of a human patient comprising:
a body dimensioned to approximate the dimensions of an implantable medical device including at least a depth, an outer perimeter and a top surface of the implantable medical device; and
a pair of recessions provided in a top surface of the body configured to receive a surgeon's fingers or a surgical tool for grasping the body of the surgical accessory.

2. The surgical accessory of claim 1 wherein the surgical accessory is further configured to fit within a ferrule adapted to receive the implantable medical device and situated in a hole formed in the cranial bone of the human patient.

3. The surgical accessory of claim 1 wherein the surgical accessory is formed form a medical grade plastic.

4. The surgical accessory of claim 1 wherein the surgical accessory is manufactured with an injection molding process.

5. A method for using a surgical accessory in a procedure involving a cranially-implantable medical device, the surgical accessory provided with a body dimensioned to approximate the dimensions of an implantable medical device including at least a depth, an outer perimeter and a top surface of the implantable medical device, said method comprising:
tracing an outline of the surgical accessory on a top surface of the patient's cranium;
forming a hole in the patient's cranium extending from an outer bone table through to an inner bone table of the cranium based on the outline;
situating a ferrule in the hole, the ferrule provided with at least one bendable tab, situating the ferrule further comprising bending the at least one tab to adjust the depth to which the ferrule extends into the hole;
placing the surgical accessory within the ferrule and assessing the extent to which a top surface of the surgical accessory is or is not approximately flush with the outer bone table; and
adjusting, if necessary, the depth of the ferrule until the top surface of the surgical accessory when placed in the ferrule is approximately flush with the outer bone table.

6. The method of claim 5, the surgical accessory further comprising a pair of recessions provided in a top surface of the body configured to receive a surgeon's fingers or a surgical tool for grasping the body of the surgical accessory as necessary while it is being manipulated relative the patient, and further comprising using the pair of recessions to manipulate the surgical accessory relative to the cranium and the ferrule.

7. A surgical accessory device for use in implanting a medical device in a cranial bone of a human patient comprising:
a body comprising a top surface, the body dimensioned to approximate the form factor of an implantable medical device; and
at least one recession provided in the top surface, the at least one recession configured to receive a surgeon's fingers or a surgical tool.

8. The device of claim 7, wherein the form factor is characterized by one or more of a depth, an outer perimeter and curvature.

9. The device of claim 7 wherein the surgical accessory is further configured to fit within a ferrule adapted to receive the implantable medical device.

10. The device of claim 7 wherein a pair of recessions are provided in the top surface.
